(19) United States
(12) Patent Application Publication
(10) Pub. No.: US 2004/0243150 A1
(43) Pub. Date: Dec. 2, 2004

(54) Skin support, apparatus and method for use in the excision of grafts

(76) Inventor: Per Gunnar Werner, Fagerstrand (NO)

Correspondence Address: CHRISTIAN D. ABEL, ONSAGERS AS, POSTBOKS 6963 ST. OLAVS PLASS, NORWAY, N-0130, NO

(21) Appl. No.: 10/204,490

(22) PCT Filed: Feb. 22, 2001
(86) PCT No.: PCT/NO01/00070

(30) Foreign Application Priority Data
Feb. 22, 2000 (NP) 20000859

(51) Int. Cl. A61B 17/50

(52) U.S. Cl. 606/132

(57) ABSTRACT

The invention relates to a skin support (19) for use in connection with the excision of grafts (2a) from a piece of skin (2), where the piece of skin (2) is attached to a frame (1) and the graft (2a) is excised from the piece of skin (2) by means of a hollow drill (9). The skin support (19) is arranged to abut against the piece of skin (2) and is movable relative to the frame (1). The skin support (19) further comprises devices for controlling the skin support's (19) movement automatically, and in such a manner that at least a section (20) of the skin support (19) is moved towards the frame (1) when the hollow drill (9) approaches the frame, the section (20) remains stationary relative to the frame (1) while the hollow drill (9) penetrates the piece of shin (2) and it is moved away from the frame (1) when the hollow drill (9) is removed therefrom.

The invention relates to a skin support for use in connection with the excision of grafts from a piece of skin, where the piece of skin is attached to a frame and the graft is excised from the piece of skin by means of a hollow drill, and the skin support is arranged to abut against the piece of skin. The invention also comprises an apparatus equipped with such a skin support together with a method for excising grafts from a piece of skin, where the piece of skin is attached to a frame and the graft is excised from the piece of skin by means of a hollow drill, where a skin support is employed which is arranged to abut against the piece of skin.

The object of the invention is the support of skin during the drilling out of grafts. The term grafts should be understood to refer to hair roots with surrounding skin which are excised in connection with the surgical transfer of hair from the nape of a patient's neck to hairless areas on the person's head. The donor skin is taken from the nape of the patient's neck, where the hair growth is strong and the hair roots are close together. The skin is excised in an approximately rectangular shape and attached to a frame, whereupon it is stretched by means of parallel displacement of one of the stretching beams in the frame, thus ensuring that it is kept relatively taut during the drill-out. Experience shows that the skin yields to the pressure from the drilling tool or the hollow drill to such an extent that it makes it difficult to cut the skin round the graft cleanly, even though the skin is well stretched.

The excised skin pieces normally have a width of 12-15 mm and may have a length of more than 100 mm. In the case in question, for drilling out the pieces of skin a wholly or semi-automatic machine is used which is programmed to drill the holes as close together as possible in the stretched skin.

The hair follicles are at varying angles to the surface of the piece of skin. The angle varies from patient to patient, but it may also vary within one and the same piece of skin. The drilling tool has to be aimed at the piece of skin at an angle corresponding to the direction of the hair follicles, in order to avoid destroying the hair follicles and the roots.

For the stretching operation a rotatable metal frame is employed, to which the piece of skin is secured in a suitable manner along the skin's longitudinal edges. The hollow drill will normally rotate at great speed while it is fed towards and through the skin at the set angle. A cylindrical graft is hereby excised which normally remains inside the hollow drill immediately behind the cutting edge, ready for removal and implantation in the skin of the head. The hollow drill's feed and rotation stops after the breakthrough, and an ejector pin is passed through the hollow drill, pushing the excised graft out of the drill and into a container. Difficulties often arise, however, in releasing the excised graft, and this will be further illustrated below.

As the drill penetrates the skin, it is pressed in the drill's direction of feed. The skin consequently forms an arc while the drilling is in progress. This creates difficulties for the drilling, particularly when the drilling angle, i.e. the angle between the drill and a perpendicular to the skin surface, is large. It will often be difficult to cut off tough fibres completely. Consequently, the graft may spin round with the hollow drill until it is pulled out of the drill and is left attached to the back of the skin. Quite thin skin fibres are spun into strong strands which are capable of pulling out the graft.

U.S. Pat. No. 5,782,843 describes a device for extracting pieces of skin with hair roots from an excised piece of skin by means of a drill. The excised piece of skin is stretched in a stretching frame whose angle can be adjusted. The device further comprises a plate part which supports the piece of skin. The piece of skin abuts against the plate part during the entire process. Fixed skin supports of this type, permanently abutting against the back of the skin, create as many problems as they solve. The back of the skin will adhere to the skin support.

Attempts have been made to remedy this by spreading friction-reducing creams on the back of the skin, but such measures are of very limited use. As the skin is perforated during the drilling operation, the skin is weakened—it becomes looser—and this reinforces the undesirable tendency for the skin to adhere to the fixed skin support. The wider the piece of skin, the greater the problem.

The problem can be remedied by a combination of a very sharp, smooth drill, a high rotation speed, suitable feed rate and a support device which supports the skin in an efficient manner.

The present invention provides the basis for a very simple and highly efficient device and method for supporting pieces of skin for use in drilling out hair grafts.

The invention comprises a skin support which is characterized in that it is movable relative to the frame and comprises devices for controlling the skin support's movement automatically, and in such a manner that at least a section of the skin support is moved towards the frame when the hollow drill approaches the frame, the section remains stationary relative to the frame while the hollow drill penetrates the piece of skin and it is moved away from the frame when the hollow drill is removed therefrom. The invention also comprises an apparatus for excising grafts from a piece of skin, comprising a frame for attaching the piece of skin which is characterized in that the apparatus comprises a skin support according to the invention. The invention finally comprises a method for excising grafts, where the piece of skin is attached to a frame and the graft is excised from the piece of skin by means of a hollow drill, and where a skin support is employed which is arranged to abut against the piece of skin. The method is characterized by moving the skin support relative to the frame and controlling the skin support's movement automatically and in such a manner that at least a section of the skin support is moved towards the frame when the hollow drill approaches the frame. The section remains stationary relative to the frame while the hollow drill penetrates the piece of skin, and the section is moved away from the frame when the hollow drill is removed therefrom.

The section of the skin support which moves backwards and forwards as described above will hereinafter be called the support section. The invention therefore comprises a support section which automatically abuts against the back of the skin before the drill reaches the skin and remains securely fixed thereto until the drill has completed the drilling and begins to withdraw. When the drill returns, the support section is automatically withdrawn from the back of the skin, thus terminating the contact between the support section and the skin. When the skin is moved to the side and upwards to the next drilling position, it moves with complete freedom relative to the support section, thereby avoiding any tearing between the rough and sticky back of the skin and the plate.

The devices for controlling the skin support relative to the piece of skin with regard to the drilling tool's movement may, for example, be implemented in the form of an electronic control system which senses the hollow drill's position, path and speed and with regard thereto controls actuators which move the skin support. The “connection” between the hollow drill's and the skin support's movements may also be implemented by means of pneumatic systems.

These devices will result in the desired effect which is the ability to excise hair grafts from the piece of skin with the least possible destruction of the skin round the graft and of the actual graft.

The stretching frame may be secured in the inclined position which best matches the direction of the hair follicles in the stretched skin. Since the angle of the hair follicles can vary over the length of the piece of skin, the frame can be programmed to change the angle as the drill is moved in the longitudinal direction of the piece of skin.

The support section's movements must be capable of being automatically adapted to the angular setting(s) selected for the stretching frame.

The support section may have a round hole through which the hollow drill is passed during drilling, thus providing optimal support round the cutting edge. Since the drill successively assumes different positions in the vertical direction, a skin support of this kind will have to be able to do likewise. This can be accomplished by means of suitable control devices.

Experiments have shown that the support section is almost equally efficient if it has a narrow slot which tightly grips the drill, permitting it to assume a number of positions in the vertical direction. The support section, however, must forcibly follow the drill's movement in the horizontal direction, this being very easy to arrange.

It will be appreciated that the piece of skin can just as well be stretched edgewise and that the support plate's slot can be located horizontally or that other stretching and processing axes may be chosen.

In a preferred embodiment of the invention, the device for automatically controlling the skin support's movement relative to the piece of skin is a substantially mechanical device.

The skin support according to a preferred embodiment of the invention comprises a support section, a rotating section and a friction section, the devices for controlling the skin support's movement relative to the frame comprising the said rotating section and friction section.

In a further preferred embodiment of the invention, the support section has a slot for the passage of the hollow drill, the slot's width being adapted to the hollow drill's diameter in order to obtain the least possible clearance between the hollow drill and the sides of the slot and the length of the slot corresponding to the hollow drill's path of movement preferably in the support section's longitudinal (vertical) direction. By this means the part of the piece of skin which is drilled through will only be supported on the sides, thus achieving support without the support section being in the way of the drill.

In a preferred embodiment of the invention the movement between the frame and the skin support is a tilting movement. For this purpose the rotating section comprises a bearing slot for receiving a pivot shaft, the pivot shaft being attached to the frame and the rotating section being able to tilt freely about the shaft in a limited angular range.

In order to restrict the support section's tilting movement relative to the frame, in a preferred embodiment of the invention the support section has a contact flange for abutment against the frame.

According to a preferred embodiment of the invention, the control of the support section's movement relative to the hollow drill is implemented by the friction section comprising a slot for the passage of a friction beam, the slot's width being adapted to the friction beam's width, thus achieving friction connection between the slot and the friction beam, and the friction beam is arranged to follow the hollow drill's movement to and from the frame, thereby transferring this movement to the skin support. However, the friction section and the friction beam are arranged to permit a certain amount of sliding between them, thus enabling the hollow drill to penetrate the skin while the support section remains stationary.

Even though the friction connection for transferring the hollow drill's movement to and from the frame to a section of the skin support is probably the best solution, it will be obvious to a person skilled in the art that the transfer can be implemented in other ways, e.g. pneumatically or by means of a control system for the parts which is connected to a computer.

Even though an embodiment of the invention will now be described where the movement of a part of the skin support towards the skin is a tilting movement, it will be understood that the invention can also be implemented by the said section being moved towards the piece of skin in many other ways, including in a straight line towards the piece of skin (where the straight line may form any angle whatever with the surface of the piece of skin) or by a rotating movement from the side of the piece of skin.

The invention will now be described by means of an example and with reference to the drawings, which illustrate a preferred embodiment and in which:

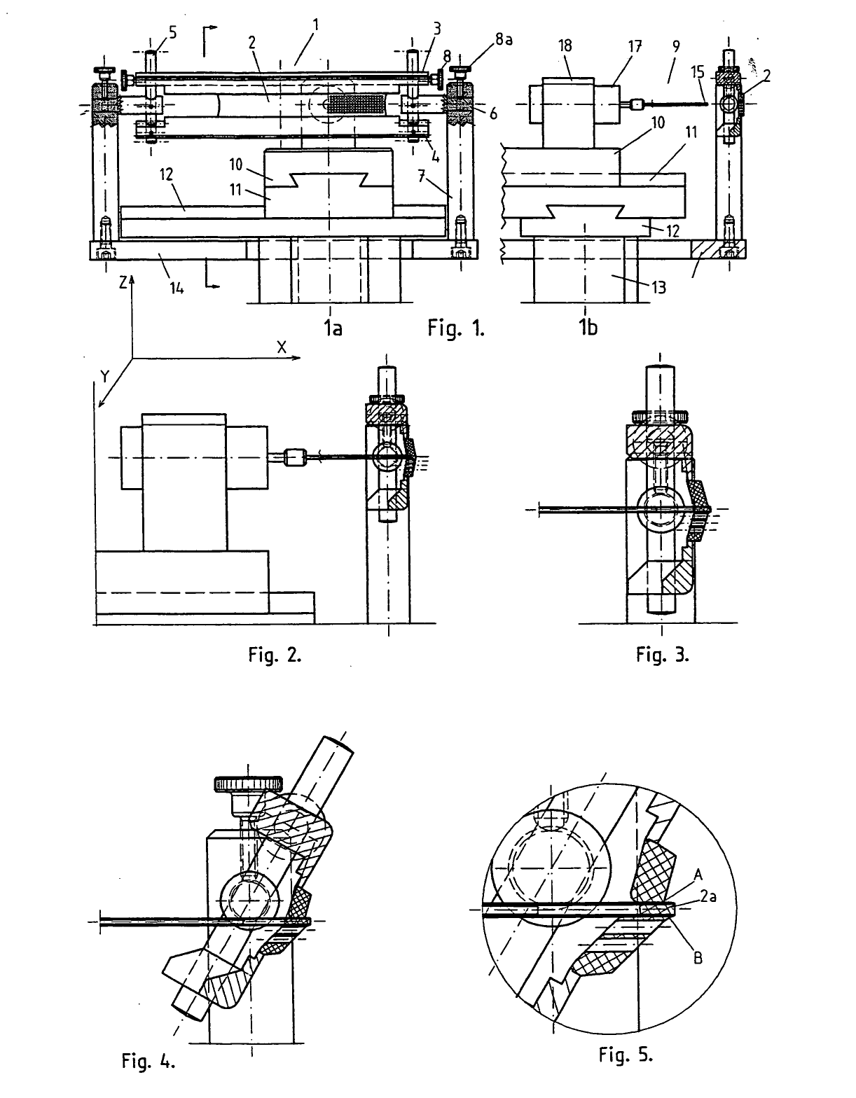

FIG. 1a illustrates an apparatus for removal of grafts according to the prior art viewed from the side and in section,

FIG. 1b illustrates the same apparatus in section before the hollow drill has reached the piece of skin,

FIG. 2 illustrates a section of the same apparatus when the hollow drill has reached the piece of skin,

FIG. 3 illustrates a section of the same apparatus when the hollow drill has cut through the piece of skin,

FIG. 4 illustrates a section of the apparatus where the frame part is angled relative to the hollow drill,

FIG. 5 illustrates a detail in FIG. 4,

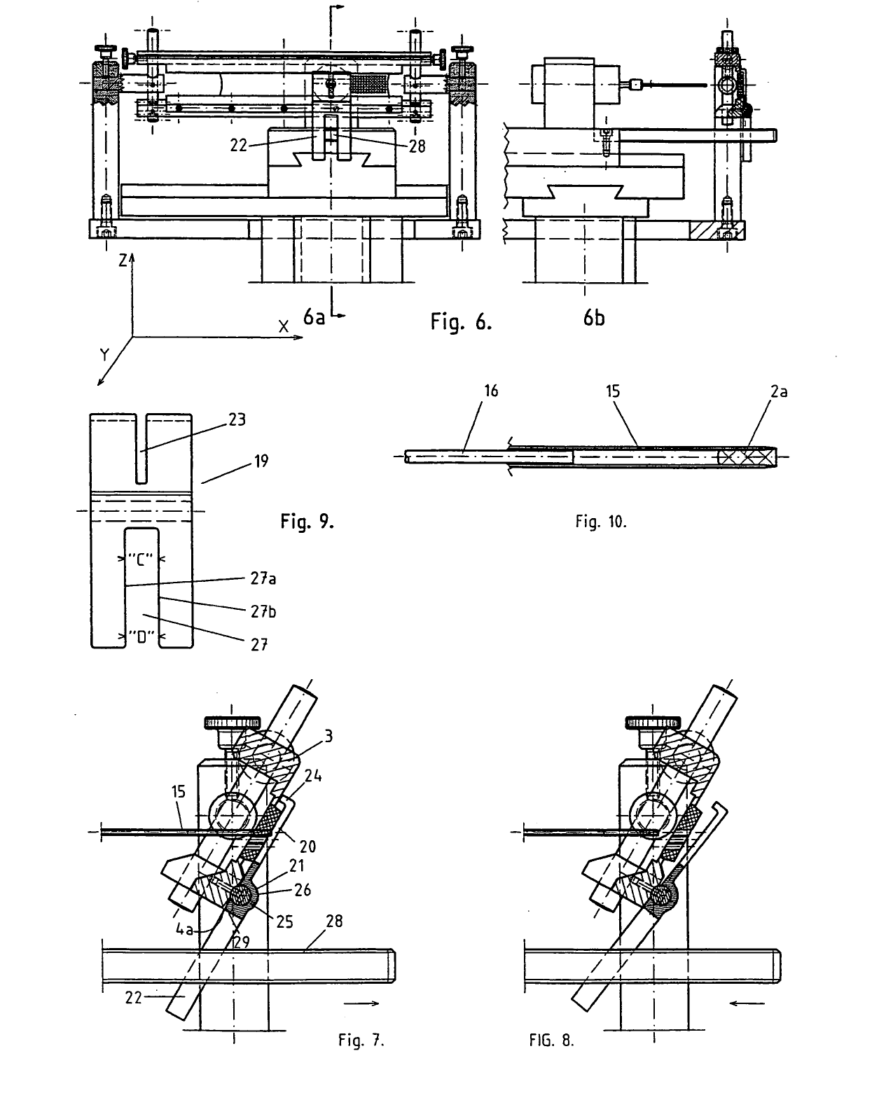

FIG. 6a illustrates an apparatus according to the invention,

FIG. 6b illustrates the same apparatus in section before the hollow drill has reached the piece of skin,

FIG. 7 illustrates the skin support according to the invention when the hollow drill penetrates the piece of skin,

FIG. 8 illustrates the skin support according to the invention when the hollow drill is removed from the piece of skin,

FIG. 9 illustrates the skin support according to the invention,

FIG. 10 illustrates the relationship between the ejector pin, the hollow drill and the graft.

The apparatus illustrated in FIGS. 1a and 1b comprises a frame 1 for receiving and stretching a strip-formed piece of skin 2, where the frame 1 comprises an upper beam 3 and a lower beam 4 together with pillar parts 5. In the frame 1 there may be defined a skin piece longitudinal direction x (corresponding to the longitudinal direction of the beams 3 and 4), a skin piece transverse direction z (corresponding to the pillars' 7 longitudinal direction) and a direction to and from the skin piece y, where the direction y forms an angle with the plane of the skin piece which is dependent on the angular position of the hair follicles in the piece of skin. The pillar parts 5 are fixed to the lower beam 4 and to the pivot shafts 6. The upper beam 3 is slidably arranged on the top parts of the pillar parts 5, and can be moved in parallel relative to the lower beam 4. The beam's 3 position can be secured by means of the adjusting screws 8. The pivot shafts 6 are rotatably mounted in the pillars 7. The frame 1 is kept in a specific angular position by means of the screws 8a. The object of this part of the apparatus is to be able to move the frame 1 with the piece of skin 2 relative to a hollow drill 9 in order to adapt the hollow drill's 9 drilling direction (y) to the directions of the hair follicles in the piece of skin 2.

The apparatus further comprises devices for movable mounting of the hollow drill 9. These devices comprise first, second and third slide parts 10, 11, 12, whereof the second and the third (11 and 12) are in the form of rails. The drilling tool 9 is fixed relative to the first slide part 10. The second slide part 11 extends along the direction y, the third slide part extends along the direction x. The slide parts are interconnected in the following manner: the first slide part 10 is movably connected to the second slide part 11, thus enabling the first part 10 to be moved over the second slide part 11 in the second part's 11 longitudinal direction. In a similar manner the second slide part 11 is movably attached to the third slide part 12 and can be moved in the third part's 12 longitudinal direction. The slide part 12 is attached to a fourth, vertical slide part 13 which can be moved vertically, i.e. in direction z. The slide part 13 is movably mounted in a slide guide fixed to the machine foundation which is not illustrated in the drawings. The base plate 14, which supports the pillars 7, is similarly fixed to the machine foundation. The foundation is not illustrated in the drawings.

The hollow drill 9 (see FIG. 10) comprises a cutting sleeve 15 which is hollow and an ejector pin 16 (FIG. 10). The hollow drill 9 is operated by an electric motor 17 which is fixed in a motor support 18 which in turn is fixed in the first slide part 10. During operation, the cutting sleeve 15 will be moved towards the frame 1 (by the slide part 10 moving along the slide part 11) while rotating, it will reach the piece of skin 2 and excise a graft 2a which will remain inside the cutting sleeve 15. The cutting sleeve's 15 rotation and feed movement stop—as explained earlier—and the ejector pin 16 is moved towards and past the cutting sleeve's 15 opening, thus expelling the graft 2a.

The cutting sleeve 15 and the ejector pin 16 are then pulled back and out of the piece of skin 2, whereupon the fourth slide part 13 raises or lowers the built-up slide parts and the motor unit to the next position.

When one row of holes has been drilled, the slide part 11 moves in the x-direction to the next hole row position, whereupon the next row of holes is drilled.

FIG. 2 illustrates in an enlarged section how the piece of skin 2 in an apparatus without skin support is pressed outwards in the hollow drill's 9 direction of movement during the drilling operation. The skin is pliable and yields to the pressure. This becomes more pronounced as the number of holes in the skin increases. FIG. 1 illustrates a situation where many holes have already been drilled and the skin has become extremely pliant. The skin's pliability is greatest when holes are drilled along the middle of the skin, i.e. furthest from the beams 3 and 4.

FIG. 3 illustrates in greater enlargement a section through the skin 2 and the hollow drill 9 just before the drill's 9 breakthrough. The skin is stretched substantially outwards, and just before the breakthrough the skin 2 becomes wrapped along the drill, thus reducing the cutting effect. The edge of the hollow drill 9 moves more and more along the skin's 2 collagen and fibres, thus making cutting difficult.

FIG. 4 illustrates the frame 4 in a commonly used setting angle of about 30 degrees. The part of the skin piece 2 located above the hollow drill 9 is almost perpendicular to the hollow drill's 9 axis, and the skin on this side above the hollow drill 9 is usually easily excised. The underlying part of the skin, however, is at a rather acute angle to the hollow drill's 9 axis, even more acute than illustrated in FIG. 2, and for this reason the edge cuts even more along the skin's fibres and collagen. In this very common situation it frequently happens that the skin's fibres and collagen are not completely excised but are spun into a thin, strong strand which, during the rotation of the drill and continued forward movement, manages to pull the graft 2a out of the hollow drill 9. This creates serious problems, since the operating personnel have to find the tiny grafts attached to the back of the skin, cut them free and further treat them manually.

FIG. 5 illustrates in even greater enlargement a section of the sectional drawing, where it is clearly illustrated at A how the hollow drill 9 has cut through the skin's fibres and the thin layer of collagen which has formed on the back of the skin. It further illustrates how fibres and collagen are pulled along with the drill 9 on its underside B. In the illustrated situation, the graft 2a spins round with the drill 9, since there is no longer anything which can restrain it. In order for the skin to be completely excised and for the graft 2a to remain inside the hollow drill, the cutting at B must take place so rapidly that the graft does not have time to spin with the drill for very many rotations.

FIGS. 6a and b illustrate an apparatus according to the invention. The reference numerals correspond to the reference numerals in FIGS. 1a and 1b. The object of the apparatus is to provide a skin support which is movable relative to the frame and where this movement is controlled with regard to the hollow drill's movement.

The skin support according to the invention will now be described in greater detail.

FIGS. 7, 8 and 9 illustrate the skin support 19, which comprises three main parts: a support section 20, a rotating section 21 and a friction section 22. The support section has a narrow slot 23 which extends in the vertical direction and a contact flange 24. The rotating section 21 has a bearing slot 25 which grips a pivot shaft 26. The friction section 22 has a slot 27 with parallel or almost parallel lateral surfaces 27a and 27b which grip a friction beam 28 in such a way that friction occurs between the slot's lateral surfaces 27a, 27b and the friction beam's lateral surfaces 28a, 28b (not illustrated in the figure).

When the hollow drill 9 is moved towards and through the piece of skin 2, the frictional force between the surfaces 27a/27b and 28a/28b results in a torque which causes the skin support 19 to rotate in an anticlockwise direction, until the contact flange 24 abuts against the top beam 3. The support section 20 abuts securely against the back of the skin, with the result that the piece of skin 2 is kept flat during drilling.

When the drill 9 is withdrawn, the friction between the surfaces 27a/27b and 28a/28b will result in a clockwise torque which causes the support section 20 to be removed from the back of the piece of skin 2. The skin support's 19 tilting movement is restricted by the fact that a part 29 of the skin support abuts against a surface 4a of the lower beam 4.

When the drilling apparatus is moved in the horizontal direction, the skin support 19 remains in the same position relative to the hollow drill 9, since the friction beam 28 is engaged with the skin support's 19 slot 27. The skin support 19 slides along the pivot shaft 26 to the new drilling position. Thus the skin support 19 will not abut against the skin when the lateral movement takes place.

The skin support 19 is made of an elastic material with a high degree of rigidity and great wear resistance. The skin support 19 may advantageously be injection moulded from POM, since this material satisfies all the requirements for mechanical strength, elasticity, wear resistance and washability as well as being unaffected by chemical disinfection and autoclaving.

The slot 27 may have uniform width, i.e. the measurements at C and D (FIG. 9) may be identical. Since the friction beam 28 acts at different distances from the pivot shaft 26 as the hollow drill 9 moves upwards and downwards, the torque will be different if the frictional force is identical at all heights. If the torque requires to be identical in positions C and D, care must be taken to ensure that the frictional force is higher at C than at D. This can be achieved, for example, by designing the slot 27 in such a way that the cross section at C is smaller than at D, or by using materials (possibly in the form of a coating) with different frictional coefficients along the slot 27.

The friction beam 28 is made of corrosion-proof material, e.g. of anodised aluminium or stainless steel. The surface of the friction surfaces 28a/28b should be polished in order to ensure the minimum of wear on the interacting surfaces.

Alternatively, the skin support 19 may be manufactured from two parts, the friction section 22 being made of, e.g., POM and the other parts or sections of metal or steel.

FIG. 10 illustrates as mentioned above an enlarged sectional drawing of the cutting sleeve 15, the graft 2a and the ejector pin 16.

The invention therefore permits a simple, clean and rapid excision of the graft from the skin. Disinfection of the equipment is also greatly simplified. The number of spoilt grafts is reduced, resulting in a more efficient treatment.

1. A skin support (19) for use in connection with the excision of grafts from a piece of skin (2), where the piece of skin (2) is attached to a frame (1) and the graft is excised from the piece of skin by means of a hollow drill (9), and the skin support (19) is arranged to abut against the piece of skin, characterized in that the skin support (19) is movable relative to the frame (1) and comprises devices for controlling the skin support's (19) movement automatically, and in such a manner that at least a section (20) of the skin support (19) is moved towards the frame (1) when the hollow drill (9) approaches the frame (1), the section (20) remains stationary relative to the frame (1) while the hollow drill (9) penetrates the piece of skin and it is moved away from the frame (1) when the hollow drill (9) is removed therefrom.

2. A skin support (19) according to claim 1, characterized in that it comprises a support section (20), a rotating section (21) and a friction section (22), the devices for controlling the skin support's movement relative to the frame (1) comprising the said rotating section (21) and friction section (22).

3. A skin support (19) according to claim 2, characterized in that the support section (20) has a slot (23) for the passage of the hollow drill (9), the width of the slot (23) being adapted to the diameter of the hollow drill (9) in order to obtain the least possible clearance between the hollow drill (9) and the sides of the slot (23), and the length of the slot (23) corresponds to the hollow drill's (9) path of movement preferably in the support section's (20) longitudinal direction.

4. A skin support (19) according to one of the preceding claims, characterized in that the rotating section (21) comprises a bearing slot (25) for receiving a pivot shaft (26), where the shaft (26) is attached to the frame (1) and the rotating section (21) can tilt freely about the shaft (26) in a restricted angular range.

5. A skin support (19) according to one of the preceding claims, characterized in that the support section (20) also has a contact flange (24) for abutment against the frame (1) with the object of restricting the support section's (20) tilting movement towards the frame (1).

6. A skin support (19) according to one of the preceding claims, characterized in that the friction section (22) comprises a slot (27) for the passage of a friction beam (28), the width of the slot (27) being adapted to the width of the friction beam (28), thus obtaining frictional connection between the slot (27a/27b) and the friction beam (28a/28b), and the friction beam (28) is arranged to follow the hollow drill's (9) movement to and from the frame (1), thereby transferring this movement to the skin support (19).

7. A skin support (19) according to claim 6, characterized in that the slot (27) and the friction beam (28) are arranged to permit a certain amount of sliding between them.

8. An apparatus for excising grafts (2a) from a piece of skin (2), comprising a frame (1) for attaching the piece of skin (2), characterized in that the apparatus comprises a skin support (19) according to one of the preceding claims.

9. A method for excising grafts (2a) from a piece of skin (2), where the piece of skin (2) is attached to a frame (1) and the graft is excised from the piece of skin by means of a hollow drill (9), where a skin support (19) is employed which is arranged to abut against the piece of skin (2), characterized by moving the skin support (19) relative to the frame (1) and controlling the skin support's (19) movement automatically and in such a manner that at least a section (20) of the skin support (19) is moved towards the frame (1) when the hollow drill (9) approaches the frame (1), the section (20) remains stationary relative to the frame (1) while the hollow drill (9) penetrates the piece of skin and the section (20) is moved away from the frame (1) when the hollow drill (9) is removed therefrom.
